(19) United States
(12) Patent Application Publication
(10) Pub. No.: US 2020/0155401 A1
(43) Pub. Date: May 21, 2020

(54) INTELLIGENT CODE CART

(71) Applicants: Fanping Wang, Encino, CA (US); Kongyuan HE, Encino, CA (US)

(72) Inventors: Fanping Wang, Encino, CA (US); Kongyuan HE, Encino, CA (US)

(21) Appl. No.: 16/719,743

(22) Filed: Dec. 18, 2019

Related U.S. Application Data
(60) Provisional application No. 62/781,480, filed on Dec. 18, 2018.

(51) Int. Cl. A61G 12/00; G06K 9/00; G10L 15/22; G16H 50/20

(52) U.S. Cl. CPC A61G 12/001; G06K 9/00335; G10L 15/22; A61H 31/005; A61G 2203/20; A61G 2203/44; A61G 2205/60; G16H 50/20

(57) ABSTRACT

The present invention is directed to an intelligent code cart for assisting a response team in performing cardiac pulmonary resuscitation. The intelligent code cart can provide graphical and audio instructions to the team based on the ACLS guidelines and also warn the team in case of any errors or omissions are observed in following the ACLS protocol. The intelligent code cart comprises a cart, a control unit, a display, one or more cameras, a speaker and input devices.

### CROSS-REFERENCE TO RELATED APPLICATIONS

This application claims the benefit of U.S. Provisional Application No. 62/781,480, entitled “Intelligent Rescue Cart with BLS/ACLS guidance and Simulation training system” filed Dec. 18, 2018, the contents of which are incorporated herein by reference in their entireties and for all purposes.

### FIELD OF INVENTION

The present invention relates to a code cart, and in particular, relates to a system and method for handing cardiac pulmonary resuscitation.

### BACKGROUND

An emergency medical condition means a sudden and, at the time, unexpected onset of a health condition that requires immediate medical treatment and/or operation. For example, a cardiopulmonary arrest is an abrupt loss of heart function and if immediate action is not taken, it can prove fatal. In-hospital cardiopulmonary arrest is common and associated with a high mortality rate. When a sudden cardiopulmonary event is discovered to have occurred to a patient in a hospital, a series of actions take place in an attempt to resuscitate that person. Optimally, a response team (referred to conventionally as a code blue response team) consisting of nurses, supervisors, respiratory personnel, technicians and a physician who, preferably, is well-versed in advanced cardiac life support (ACLS) procedures, is summoned to undertake the resuscitation procedures.

Advanced cardiac life support (ACLS) refers to a set of clinical guidelines for the primary treatment of cardiac arrest, stroke, myocardial infarction, and other life-threatening cardiovascular emergencies. The guideline is published by the American Heart Association and the International Liaison Committee of the Resuscitation. The current ACLS guidelines are set into several groups of “algorithms”—a set of instructions that are followed to standardize treatment and increase its effectiveness. These algorithms usually come in the form of a flowchart, incorporating ‘yes/no’ type decisions, making the algorithm easier to memorize.

Under emergency conditions, expeditious access to medicines and equipment is crucial. The crash cart was an important invention by the ECRI Institute that helped convey and dispense emergency medication/equipment at the site of medical/surgical emergency for life support protocols (ACLS/ALS) to potentially save someone's life. The cart, also known as a code cart or crash trolley carries the equipment for cardiopulmonary resuscitation and other medical supplies while also functioning as a support litter for the patient. The cart is typically a set of trays/drawers/shelves on wheels.

Although the response team consisting of nurses, supervisors, respiratory personnel, technicians and a physician should be well-versed in advanced cardiac life support (ACLS) procedures. Most nursing personnel who work on a typical hospital floor are not required to learn or be certified in ACLS. Furthermore, most physicians outside of an intensive care unit (ICU) or emergency room (ER) do not use the ACLS protocols often enough to retain the precise knowledge of the many medications and specific timing sequences involved in a code blue situation. ACLC cards may not be handy in an emergency or updated protocol may not be available. Apparatus are known in the art that overcomes the above problem, for ex., For U.S. Pub. Application 20080125821 discloses a hospital crash cart incorporating an ACLS method and apparatus operable to guide responders, via audible prompts and visual cues, through the proper procedures to be applied to a patient during a cardiac arrest. The apparatus is in the form of a computer with a display fixed to the crash cart.

Known apparatus suffer from one or more drawbacks. The major drawback of the known apparatus is that they required to be manually set by the response team. The response team generally interacts with the apparatus through input means, such as touch screen, mouse, keypad and like. Firstly, details of the patient are entered and then the required ACLS protocol is determined. The apparats typically function as a digital version of the ACLS guidelines. The team can refer to the desired information from the apparatus like a normal computer. A need is appreciated for an automated system that requires minimum input from the response team and can assist a response team in the procedure of cardiopulmonary resuscitation.

Hereinafter, the phrase “cardiac pulmonary resuscitation” is also referred to as a procedure. The term “response team” used hereinafter connotes any medical team charged with performing the cardiac pulmonary resuscitation in a hospital. The term “attendant” used hereinafter connotes a member of the response team and includes nurses, supervisors, respiratory personnel, technicians and a physician.

### SUMMARY OF THE INVENTION

The principal objective of the present invention is therefore directed to an intelligent code cart that could assist the response team in performing cardiopulmonary resuscitation in a hospital.

Another objective of the present invention is that the intelligent code cart provisions conveying and dispensing of medical supplies.

Still another objective of the present invention is that the intelligent code cart reduces human error and knowledge gaps during cardiopulmonary resuscitation.

Still another objective of the present invention is that the intelligent code cart enhances the efficiency of the response team in cardiopulmonary resuscitation.

Still another objective of the present invention is that the intelligent code cart provides for the calculation of doses.

Still another objective of the present invention is that the intelligent code cart requires minimum input from the response team during cardiopulmonary resuscitation.

Still another objective of the present invention is that the intelligent code cart could retrieve patient records and as well update the patient record with the details of the intervention.

Still another objective of the present invention is that the intelligent code cart retrieves patent diagnostic/monitoring data from the patient monitoring system.

Still another objective of the present invention is that the intelligent code cart aids the response team to locate medicine and equipment arranged with in the apparatus.

Still another objective of the present invention is that the intelligent code cart records the procedure for later analysis and training.

Still another objective of the present invention is that the intelligent code cart provides visible and audible alarms appropriate to various unsafe conditions.

Still another objective of the present invention is that the intelligent code cart provides simulated training of ACLS protocols.

Still another objective of the present invention is that the intelligent code cart maintains a running inventory of the medical supplies contained in the apparatus.

Yet another objective of the present invention is that the intelligent code cart provides audible instructions to the response team.

Further objects and advantages will become apparent from a consideration of the ensuing description and the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a block diagram showing different component of the intelligent code cart, in accordance with an embodiment of the present invention.

FIG. 2 is a block diagram showing the intelligent code cart in communication with patient records and patient monitoring system, in accordance with an embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

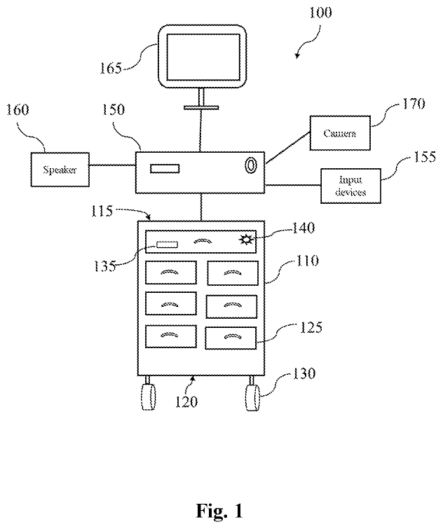

Referring to FIG. 1, which is a block diagram showing different components of the intelligent code cart 100 in accordance with an embodiment of the present invention. As shown in FIG. 1, the intelligent code cart comprises a cart 110 for conveying and dispensing medical supplies and equipment. The structure and functioning of the cart 110 are known in the art for supplying and dispensing of medical supplies for a procedure in a hospital. The cart 110 includes a top platform 115, a bottom platform 120 and a central storage section intermediately positioned between the top platform 115 and the bottom platform 120. Multiple drawers and shelves 125 are provided in the central storage section. Preferably, the drawers 125 are organized according to the ACLS guidelines. The contents of the cart 110 are organized in a manner which reflects current ACLS guidelines drawer by drawer, in a left-to-right fashion. Equipment includes Monitor/defibrillators, suction devices, and bag valve masks (BVMs) of different sizes. The defibrillator can be positioned on the top platform 115 or mounted on the side of the cart 110. The cart 110 is supported on four or more castor wheels 130 mounted at underside of the bottom platform 120. Each drawer/shelf 125 could have a label 135 printed thereon, for example, Arabic numerals could be printed on the front face of each of the drawers 125. These labels 135 can be used to identify the drawer, for example, a particular medicine is stored in the 10th drawer. The 10th drawer can be quickly identified by the label printed thereon. Suitable handles coupled to the drawers help to slide open the drawer. A list of items contained in a drawer could also be provided on the front face of the respective drawer. Such a list could be a printed list pasted on the drawer or an LCD. Further, an LED indicator 140 can be provided on each of the drawers 125 and the LED indicator 140 is electrically connected to a control unit 150. The LED indicator 140 functions to indicate the respective drawer by blinking.

The control unit 150 shown in Fig .1 comprises a processor and a memory enclosed in a cabinet. The control unit 150 can be installed in the central storage section of the cart 110 itself or can be positioned over the top of the upper platform 115 of the cart 110. The control unit 150 is programmed with the ACLS guidelines including the algorithms. The programming is such that the control unit 150 can analyze the ACLS guideline and provide visible and audible instructions to the response team in performing the cardiopulmonary resuscitation. In another embodiment, the control unit 150 can also be configured to connect with the medical equipment contained in the cart 110. In one case, the control unit 150 is connected to a blood pressure monitor, pulse oximeter and EKG. Also, the control unit 150 can be connected to any patient monitoring system used in the procedure. The control unit 150 in a preferred embodiment can be configured to retrieve the readings from the medical equipment.

In another embodiment, the control unit 150 can also be coupled to one or more of the drawers 125 of the cart 110. One or more of the drawers 125 can be configured with an electric motor to slide open the drawer 125. The electric motor can be triggered by the control unit 150 causes the drawer 125 to pop out. For example, the drawer containing an intended medicine, to be given to the patient, can be opened by the control unit 150. Alternatively, the control unit 150 can also trigger the LED indicator 140 on the drawers 125 to indicate the drawer which has the intended medicine.

In another embodiment, the control unit 150 can also be configured with an inventory management software. The control unit 150 can maintain a running inventory of the contents of the cart 110. The inventory management software can retrieve data from multiple sensors coupled to the cart. The sensors configured to track the withdrawal of the medicines and the medical equipment form the cart 110. In one embodiment, RFID tags can be coupled to the drawers 125 which can detect the withdrawal of medicines from the drawers 125. This data related to the withdrawal of medicines can be populated in the inventory management software. In another embodiment, an RFID reader can be coupled to the cart 110, such that an attendant withdrawing the medicine from a drawer can scan the medicine with the RFID reader. The same can then be updated in the inventory management software. Besides managing the inventory, the control unit 150 can also track the interventions given to the patient based on the medicine withdrawn from the cart 110.

Further shown in FIG. 1 is that the control unit 150 is connected to one or more input devices 155. The input devices 155 can be used to program and instruct the control unit 150. In one case, the input devices include a microphone. The microphone can be used to give voice commands to the control unit 150, for example, an attendant can orally ask the intelligent code cart 100 about the dose of epinephrine to be administered to the patient by saying “what is the dose of epinephrine?” The control unit 150 can analyze the verbal inputs using one or more known algorithms. The functioning of such algorithms/software is known to a skilled person for interpreting voice data. Examples of such algorithms include Siri, Alexa, and Cortana. To make the interpretation of voice commands more accurate, the control unit 150 can be configured to respond to only limited vocabulary. The response team can learn the vocabulary of the intelligent code cart 100 to verbally interact with the intelligent code cart 100. For example, “ok cart” could trigger the voice input software of the control unit 110. Thereafter, another voice command can be given to the control unit 150, for example, “dose of epinephrine.”

In one case the input means can include a mouse, a keyboard, a touch interface and like. Different kinds of input devices for use with computers are known in the art and such input devices are within the scope of the present invention. Further can be seen in FIG. 1 that the control unit 150 is connected to a speaker 160. The intelligent code cart 100 can broadcast voice instructions and alerts to the response team through the one or more speakers 160.

A display 165 is shown to be coupled with the control unit 150. The display 165 can be a monitor, such as LCD monitor. The display can be mounted over the upper side of the top platform 115. The display 165 can show graphical instruction, prompts, alerts, and other information to the response team. Also, the display can be configured with a touch interface for interacting with the intelligent code cart 100. The structure and functioning of the tough interfaces coupled to a display are known to a skilled person for interacting with a computer.

The intelligent code cart 100 further comprises one or more cameras 170 connected to the control unit 150. The camera170 can be mounted over the upper side of the top platform 115 or side of the cart 110. Preferably, the camera 170 can be coupled to the cart 110 through a revolving arm which allows the camera 172 to be adjusted in an intended direction. The control unit 150 can adjust the positioning of the camera 170 to focus on the procedure. Thus, camera 170 can automatically position itself in the intended direction irrespective of the positioning of the intelligent code cart 100. The control unit 150 can also be configured to record the procedure and store the recordings in the memory of the control unit 150. The recordings can be useful for any analysis or training purposes. Furthermore, the control unit 150 can be configured with an image recognition algorithm to recognize the actions and gestures of one or more attendants. For example, the control unit 150 can recognize that an attendant is giving an injection to the patient or another attendant performing cardiac compressions to the patient.

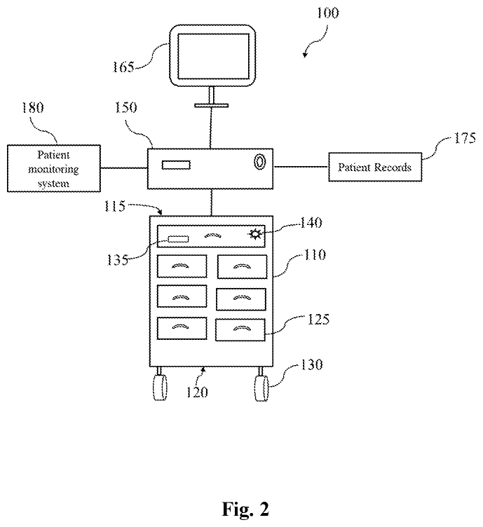

FIG. 2 is a block diagram showing the intelligent code cart 100 connected to a patient record database 175 and a patient monitoring system 180. The patient record database 175 contains records of the patients, wherein each record has an identifier. The control unit 150 can automatically retrieve a record of the patent undergoing cardiopulmonary resuscitation. Moreover, the record of the patient could also be updated with details of the procedure and any intervention given to the patient. This prevents any gaps or omissions in the patient record. In one embodiment, the control unit 150 can also be configured to retrieve readings from a patient monitoring system, for example, the control unit can retrieve the data from ECG, defibrillators, heart rate monitors, oximeters, etc.

In one embodiment, the intelligent code cart 100 of the present invention can be further equipped with simulation training software. The software has a multi-functional simulation training module, which can be used for routine training of the attendants and other hospital staff. So, the response team can get used to the application of intelligent code cart 100 and achieve clinical re-education and training.

In another embodiment, the present invention is directed to a method for assisting a response team in performing cardiopulmonary resuscitation. When a patient reports signs of cardiopulmonary arrest, such as cardiac pain, difficulty in breathing, etc. The response team can be immediately activated to handle the emergency. Meanwhile, the intelligent code cart 100 of the present invention can also be activated. The intelligent code cart 100 on activation can try to identify the patient reporting signs of cardiopulmonary arrest. In case, the record of the patient is available in the patient records 175, the intelligent code cart 100 can retrieve the said record which may be having details of the patient, such as name, weight, etc. In case, the intelligent code cart 100 could not automatically identify the patient, a prompt can be generated by the intelligent code cart 100 and an attendant can input the patient id using the keyboard 155 connected to the control unit 150. The control unit 150 can then retrieve the patient record linked to the entered patient id. Still, if the patient record could not be found by the intelligent code cart 100, the intelligent code cart 100 may prompt the attendant to enter the basic details of the patient, such as the weight of the patient. The weight of the patient can be used to calculate the dose of medicine to be administered to the patient. Firstly, the response team can determine the blood pressure and optionally other parameters, such as pulse and blood oxygen level. The intelligent code cart 100 can retrieve the above determinations. Alternatively, the same could be fed into the intelligent code cart 100 by any attendant. Moreover, other signs of the patient can also be fed to the intelligent code cart 100 by the attendant, for example, the patient is having difficulty in breathing.

Thereafter, the intelligent code cart 100 can trigger the cameras 170 to capture the procedure. Based on the observation fed by the attendant, the intelligent code cart 100 can display a particular ACLS protocol to the response team to be followed. Alternatively, the intelligent code cart 100 can provide a list of ACLS protocols most suited to the present situation. The response team can then select the intended ACLS protocol from the list. The intelligent code cart 100 can then initiate the ACLS protocol by providing both verbal and graphical instructions to the response team. The intelligent code cart 100 can calculate the dose of intervention to be given to the patient and instruct the response team to administer the determined dose of the intervention. The intelligent code cart 100 can indicate the location of the medicine by blinking the indicator 140 on the drawer containing the intended medicine. Alternatively, the intelligent code cart 100 can pop open the drawer containing the intended medicine. This prevents any wastage of time in administering the intervention.

In one embodiment, intelligent code cart 100 can track the procedure and actions of the response team. The intelligent code cart 100 through the images of the procedure captured by the camera 170 can recognize the actions and gestures of the attendants. Moreover, this data can be corroborated with other data, for example, data from sensors coupled to the drawers and patient monitoring system. In one case, the withdrawal of medicine from the drawers as detected by the sensor coupled to the drawer can be corroborated with the action of the attendant administering the intervention to the patient. Moreover, the effect of the intervention can be determined by the intelligent code cart 100 through medical equipment or patient monitoring system. Alternatively, the same could be fed to the intelligent code cart 100 by an attendant. Thus, the intelligent code cart 100 can track the whole transaction of instructing the administration of an intervention, withdrawal of medicine from the cart and administration of intervention to the patient by the attendant.

The intelligent code cart 100 through the above-tracked details, can identify any omission of a step of a protocol or if the response team is not following the protocol correctly. In case, any such observation is made by the intelligent code cart 100, the intelligent code cart 100 can warn the response team with details of the omission or any errors in following the protocol. Alerts both graphical and audio can be broadcasted to the response team. The details of the interventions given to the patient can then be updated in the patient record by the intelligent code cart 100. Moreover, the recordings of the procedure can be stored for later analysis and training.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

What is claimed is:

1. A method of assisting a response team in performing cardiopulmonary resuscitation, the response team comprising one or more attendants, the method comprising:
providing an intelligent code cart, the intelligent code cart comprising:
a control unit comprising a processor and a memory,
a plurality of drawers, the plurality of drawers comprising medicines and
medical equipment, the plurality of drawers coupled to the control unit,
one or more cameras coupled to the control unit,
a display coupled to the control unit, and
one or more input devices coupled to the control unit;
receiving, by the intelligent code cart, first details of a patient;
identifying, by the intelligent code cart, a standard protocol for performing the cardiopulmonary resuscitation;
receiving, by the intelligent code cart, from the one or more cameras, a sequence of images, the sequence of images is of the response team performing the cardiopulmonary resuscitation;
analyzing, by the intelligent code cart, the sequence of images to recognize actions and gestures of the response team;
providing step by step instructions, by the intelligent code cart, to the response team, the step by step instructions based on the standard protocol for performing the cardiopulmonary resuscitation;
observing, by the intelligent code cart, any error in following the standard protocol by the response team; and
issuing alerts, by the intelligent code cart, to the response team, based on the error.

2. The method of claim 1, wherein the first patient details are retrieved by the intelligent code cart from a patient record database.

3. The method of claim 1, wherein the first patient details are received, by the intelligent code cart, from the one or more attendants.

4. The method of claim 1, wherein the first patient details comprises weight of the patient, and the method further comprises a step of calculating dose of an intervention based on the weight of the patient.

5. The method of claim 1, wherein the step of identifying a standard protocol further comprises steps of:
displaying, by the intelligent code cart, to the response team, a list of standard protocols, the list of standard protocols based on the first patient details and observed signs of the patient; and
selection, by the response team, of the standard protocol from the list of the standard protocols.

6. The method of claim 1, wherein the standard protocol is based on ACLS guidelines.

7. The method of claim 1, wherein the method further comprises a step of updating a patient record with details of interventions admitted to the patient.

8. The method of claim 1, wherein the method further comprises a step of receiving, by the intelligent code cart, details of withdrawal of an intended medicine from one of the plurality of drawers.

9. The method of claim 8, wherein the method further comprises a step of:
receiving, by the intelligent code cart, an action of the administration of the intended medicine to the patient based on analysis of the sequence of images; and
corroborating the details of the withdrawal of the intended medicine to the action of the administration of the intended medicine.

10. The method of claim 1, wherein the method further comprises a step of guiding the response team to one drawer of the plurality of the drawers by blinking an indicator coupled to the one drawer, the one drawer containing a medicine to be administered to the patient.

11. The method of claim 1, wherein the method further comprises a step of opening one drawer of the plurality of the drawers by the control unit, wherein the one drawer is having a medicine instructed by the intelligent code cart for administration to the patient.

12. The method of claim 1, wherein the step of observing any error in following the standard protocol is based on comparison of the actions of the response team with the standard protocol.

13. The method of claim 1, wherein the plurality of images is a video and the method further comprise a step of storing the video in the memory of the control unit.

14. The method of claim 1, wherein the step by step instructions are displayed on the display and broadcasted through the speaker.

15. The method of claim 1, wherein the method further comprises a step of retrieving, by the control unit, data from the medical equipments, the data related to a condition of the patient determined by the medical device.

16. An intelligent code cart comprising:
a control unit;
a central storage section, the central storage section comprises a plurality of drawers, the plurality of drawers coupled to the control unit;
one or more medical equipment, the one or more medical equipment mounted to the central storage section, the one or more medical equipment in electrical communication with the control unit;
a plurality of indicators coupled to one or more of the plurality of drawers, the plurality of indicators configured to be triggered by the control unit to blink;
one or more sensors coupled to the one or more of the plurality of drawers, the one or more sensors configured to detect withdrawal of a medicine from a drawer;
one or more cameras coupled to the control unit;
a display mounted on top of the central storage section, the display coupled to the control unit;
a speaker coupled to the control unit; and
one or more input devices coupled to the control unit;
wherein the control unit is configured to:
detect withdrawal of the medicine from the drawer,
capturing a sequence of images of the response team by the one or more cameras, and
recognize actions and gestures of the response team from the sequence of images.

17. The intelligent code cart of claim 1, wherein the intelligent code cart further comprises a motor coupled to the plurality of drawers, wherein the motor can be triggered by the control unit to open one of the plurality of drawers.

18. The intelligent code cart of claim 1, wherein the one or more input devices includes a microphone, the controlling unit is configured to interpret voice commands received through the microphone.

19. The intelligent code cart of claim 1, wherein the display is having a touch input interface.

20. The intelligent code cart of claim 1, wherein the one or more sensors are RFID based.
